(19) United States
(12) Patent Application Publication
(10) Pub. No.: US 2025/0204830 A1
(43) Pub. Date: Jun. 26, 2025

(54) FLEXIBLE DRY ELECTRODES

(71) Applicant: NTT RESEARCH, INC., Sunnyvale, CA (US)

(72) Inventors: George AL BOUSTANI, Sunnyvale, CA (US); Tetsuhiko TESHIMA, Sunnyvale, CA (US); Bernhard WOLFRUM, Sunnyvale, CA (US); Lukas HIENDLMEIER, Sunnyvale, CA (US)

(73) Assignee: NTT RESEARCH, INC., Sunnyvale, CA (US)

(21) Appl. No.: 18/849,203

(22) PCT Filed: Mar. 21, 2023
(86) PCT No.: PCT/US2023/064768

§ 371 Date: Sep. 20, 2024

Related U.S. Application Data
(60) Provisional application No. 63/322,534, filed on Mar. 22, 2022.

(51) Int. Cl. A61B 5/263

(52) U.S. Cl. CPC A61B 5/263; A61B 2562/0209; A61B 2562/0285; A61B 2562/04; A61B 2562/125

(57) ABSTRACT

Embodiments disclosed herein provide 3-dimensional dry electrodes formed by a plurality of 2-dimensional hair electrodes. Each of the hair electrodes may be formed by non-conductive hair electrode base and a conductive layer. The non-conductive hair electrode base may be formed of substances such as a polymer using techniques such as laser cutting. A conductive layer may be applied to the hair electrode bases using technique such as metal sputtering to generate the plurality of 2-dimensional hair electrodes. The plurality of 2-dimensional hair electrodes may then be grouped together (e.g., rolled) to form a 3-dimensional dry electrode.

### PRIORITY

This application claim priority to U.S. Provisional Application No. 63/322,534, filed Mar. 22, 2022 and entitled “Flexible Dry Electrodes,” which has been incorporated in its entirety by reference.

### BACKGROUND

A widespread clinical diagnostic technique is to measure electrical current through skin attached electrodes. The measured currents are processed for non-invasive biophysical tracking of vital organ activity—the skin-detected electrical activity correlates with the biophysical activity of the vital organ (e.g., heart rate). Some examples of such skin electrode based clinical tracking include electro-cardiogram (ECG) and electro-encephalogram (EEG). The popular skin electrodes are wet electrodes, which use electrogel to enhance the electrical impedance between the electrode and the skin. FIG. 1A shows an example of a wet surface electrode 100. As shown, the wet surface electrode 100 uses an electrogel 104 to enhance the impedance between the wet surface electrode 100 and the skin 102. For example, in a widely used silver (Ag) or silver chloride (AgCl) skin electrode, electrogel is used to generate an impedance range of 0.1 kΩ to 3 kΩ.

Wet electrodes, however, have several disadvantages. Wet electrodes require a long implementation time—having the preparation steps of injecting the electrogel with syringe and spreading the electrogel for each wet electrode (a typical clinical diagnostic technique may use 32 to 256 wet electrodes). Furthermore, electrogel is prone to drying, thereby making the wet electrodes unsuitable for long-term applications and/or experiments. To mitigate these disadvantages, dry electrodes have been developed. For example, FIG. 1B shows an example dry electrode. As shown, a dry electrode 106 slightly pierces the outer layer of the skin 102 and the electrode 106-skin 102 impedance is based on the available electrolyte concentration underneath the skin 102 layers. But conventional dry electrodes have not been able achieve the requisite clinical performance level.

As such, a significant improvement on the electrode technology, particularly dry electrode technology is therefore desired.

### SUMMARY

Embodiments disclosed herein may solve the aforementioned technical problems and may provide other solutions as well. An example electrode may be formed by grouping together a plurality of hair electrodes (also referred to as bristle electrodes). Each of the hair electrodes may include a non-conductive hair electrode base that may provide the structural integrity and mechanical properties of the hair electrode. The hair electrode base may be formed by sculpting a substrate, e.g., laser cutting a polymer into hair structures. A conductive layer may be provided around the hair electrode base. The conductive layer may be formed by sputtering a metal (e.g., gold) on the hair electrode base.

In an embodiment, a flexible electrode for a clinical equipment and configured to detect electrical signals from a live tissue may be provided. The flexible electrode may comprise a plurality of hair electrodes, wherein each of the hair electrodes comprises: a non-conductive and flexible hair electrode base defining the structure of the hair electrode; and a gold outer layer covering the electrode base, the gold outer layer forming a conductive portion of the hair electrode and configured to detect the electrical signals from the live tissue.

In another embodiment, a flexible electrode configured for a clinical equipment and configured to detect electrical signals from a live tissue may be provided. The flexible electrode may comprise a plurality of hair electrodes, wherein each of the hair electrodes comprises: a non-conductive and flexible hair electrode base defining the structure of the hair electrode; and a conductive polymer outer layer covering the electrode base, the conductive polymer outer layer forming a conductive portion of the hair electrode and configured to detect the electrical signals from the live tissue.

In yet another embodiment, a method of manufacturing a flexible electrode for a clinical equipment and configured to detect electrical signals from a live tissue may be provided. The flexible electrode may comprise a plurality of hair electrodes. The method may comprise fabricating a plurality of non-conductive hair electrode bases for the plurality of hair electrodes; sputtering a metal on the plurality of hair electrode bases to form the conductive portions of the plurality of hair electrodes; and rolling the plurality of hair electrode bases with the sputtered metal to form the flexible electrode.

### BRIEF DESCRIPTION OF DRAWINGS

FIG. 1A shows an example prior art wet surface electrode.

FIG. 1B shows an example prior art dry electrode.

FIG. 2 shows an illustrative process of fabricating a 2-dimensional hair electrode, according to example embodiments of the present disclosure.

FIG. 3A-3E show example shapes of 2-dimensional hair electrode, according to example embodiments of the present disclosure.

FIG. 4A shows an illustrative process of fabricating a 3-dimensional electrode, according to example embodiments of the present disclosure.

FIG. 4B shows examples of different shaped or sized bristles rolled into different shapes, according to example embodiments of the present disclosure.

FIGS. 5A-5D show illustrations of 3-dimensional electrodes, according to example embodiments of the present disclosure.

FIG. 6 shows an illustrative circuit using the 3-dimensional electrodes, according to example embodiments of the present disclosure.

FIG. 7 shows a graph showing impedance characteristics of an illustrative 3-dimensional electrode compared to impedance characteristics of a conventional wet electrode, according to example embodiments of the present disclosure.

FIG. 8 shows a flow diagram of an illustrative method of manufacturing a 3-dimensional electrode, according to example embodiments of the present disclosure.

FIGS. 9A-9B show an example holder for a 3-dimensional electrode, according to example embodiments of the present disclosure.

FIG. 10A-10B show an example process of using a holder with the 3-dimensional electrode, according to example embodiments of the present disclosure.

FIG. 10C shows an example method of using the holder with the 3-dimensional electrode, according to example embodiments of this disclosure.

FIGS. 11A-11B show an example fastening member of a holder of a 3-dimensional electrode, according to example embodiments of the present disclosure.

FIGS. 12A-12B show an example screw holder of a holder of a 3-dimensional electrode, according to example embodiments of the present disclosure.

FIG. 13 shows an example chart illustrating a comparison of the 3-dimensional electrodes, according to example embodiments of the present disclosure, with conventional dry and wet electrodes.

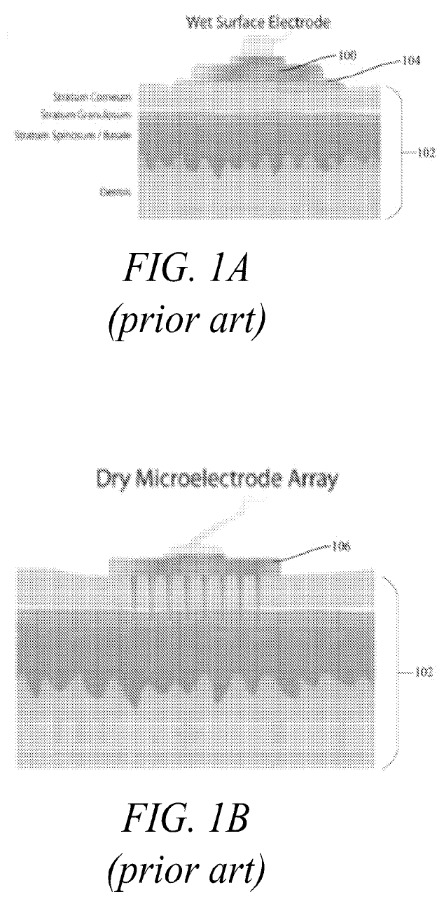

The figures are for purposes of illustrating example embodiments, but it is understood that the present disclosure is not limited to the arrangements and instrumentality shown in the drawings. In the figures, identical reference numbers identify at least generally similar elements.

### DESCRIPTION

Embodiments disclosed herein provide 3-dimensional dry electrodes formed by a plurality of 2-dimensional hair electrodes. Each of the hair electrodes may be formed by non-conductive hair electrode base and a conductive layer. The non-conductive hair electrode base may be formed of substances such as a polymer using techniques such as laser cutting. A conductive layer may be applied to the hair electrode bases using technique such as metal sputtering to generate the plurality of 2-dimensional hair electrodes. The plurality of 2-dimensional hair electrodes may then be grouped together (e.g., rolled) to form a 3-dimensional dry electrode.

It should be noted that the term “2-dimensional” is used to describe a generally flat structure, which of course exists in the three dimensional world with length, width, and depth, but because it is generally flat, the depth is much less than the length and width, and is referred to in this description as “2-dimensional” when in fact it is three dimensional, but only to describe it in contrast to a rolled up example, where a long, flat sheet of electrode bristles is rolled up as described herein, thereby making the overall dimensions of the rolled up system a more three dimensional look, although of course, they all exist in the three dimensional real world.

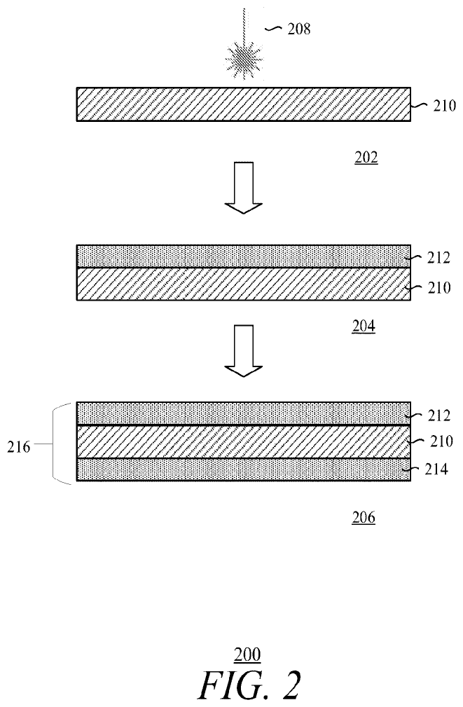

FIG. 2 shows an illustrative process 200 of fabricating a 2-dimensional hair electrode, according to example embodiments of this disclosure. The 2-dimensional electrode may form a single strand within a 3-dimensional electrode structure. The process 200 is illustrated as includes steps 202, 204, and 206, which are provided as just examples and should not be considered limiting. Processes with alternative, additional, or fewer number of steps are to be considered within the scope of this disclosure. Furthermore, the steps 202, 204, and 206 are merely for identification and is meant neither to convey a limitation to the shown discrete steps nor a limitation to the shown sequence of the steps.

At step 202 an electrode base 210 for the 2-dimensional hair electrode may be formed. The hair electrode base 210 may be the non-conducting part of the 2-dimensional hair electrode and may provide structural integrity and determine the overall shape of the 2-dimensional hair electrode (e.g., circular cross section, rectangular cross section). The hair electrode base 210 may also largely govern the mechanical properties of the 2-dimensional hair electrode. The mechanical properties may include stress and strain, which may determine the flexibility of the 2-dimensional hair electrode when pressed against the skin.

Hair electrode base 210 base may be formed of any type of material such as polymer, cellulose, chitin, chitosan, synthetic flexible fiber, microfiber, nanotube, parylene, elastomer, thermal elastomer, liquid crystal elastomer, Polysacharides, and/or natural fabrics, or any combination of any of the above in any permutation. It should be understood that these materials are just some examples and other substances forming the hair electrode base 210 should also be considered within the scope of this disclosure.

Using a substrate, e.g., one formed from one or more of the aforementioned materials, the hair electrode base 210 may be formed. For example, electromagnetic radiation such as a laser 208 may be used to generate, sculpt, and/or shape the hair electrode base 210 out of the substrate. As an example, the laser 208 may sculpt or shape a plastic material such as a polyimide into a hair structure, which may then form the hair electrode base 210. The laser 208 is just but an example, and other types of technology may be used to generate and/or shape the hair electrode base 210. For instance, liquid cutting mechanisms such as a water jet cutting may be used. Alternatively or additionally, solid cutting mechanisms such as diamond cutting may be used. The hair electrode base 210 may also be generated through molding, such as injection molding. Another technology to generate the hair electrode base may be 3D printing. Therefore, any type of technology forming a strand like structure, which may then form the hair electrode base 210 should be considered within the scope of this disclosure. The hair electrode base 210 may be formed as a pillar structure (e.g., having a non-hollow cross section) or a tube (e.g., having a hollow cross section), or rectangular structure.

At steps 204 and 206, the hair electrode base 210 may be coated with a first conductive material layer 212 and a second conductive material layer 214 to generate the 2-dimensional hair electrode 216. In some embodiments, the steps 204 and 206 may be sequential: the hair electrode base 210 may be coated with a first conductive material layer 212 on one side and then coated with a second conductive material layer 214 on the other side. In other embodiments, the steps 204 and 206 may not be sequential in that the hair electrode base 210 may be coated both conductive material layers 212 and 214 at the same time. It should be understood, however, that FIG. 2A shows longitudinal cross sections of the 2-dimensional hair electrode as it is formed—and while the longitudinal cross section defines the two conductive material layers 212 and 214, it should be understood that the actual shape of the 2-dimensional hair electrode may be, for example, cylindrical or cubic, and the conductive material may be coated on all the external surfaces of the 2-dimensional hair electrode. Furthermore, the term 2-dimensional hair electrode may be used to distinguish this electrode from the eventually formed 3-dimensional electrode, which may be formed by a plurality of 2-dimensional hair electrodes. Therefore, the term 2-dimensional hair electrode covers, as described above, any type of hair electrode having all three spatial dimensions (length, width, and height).

Any type of technology may be used for the adding the conductive material layers 212 and 214 onto the hair electrode base 210. For example, for conductive materials 212 and 214 may be formed by a metal (e.g., gold), the metal may be sputtered to the hair electrode base 210. Alternatively or additionally, the metal may be thermally and/or electrochemically deposited onto the hair electrode base 210. In some embodiments, the metal may be into a paste, and the hair electrode base 210 may be dipped into the paste to receive the metal coating. In some embodiments, the conductive material layers 212 and 214 may be formed by metal nanotubes or metal nanoparticles. The metal may also be formed into the powder, and the powder may be pressed into the hair electrode base 210.

In some embodiments, the conductive material layers 212 and 214 may be formed by conductive polymers. The conductive polymers may be electrochemically deposited to the hair electrode base 210. Alternatively or additionally, the conductive polymers may be formed into a paste, and the hair electrode base 210 may be dipped into the paste thereby receiving the coatings as the conductive layers 212 and 214. The conductive polymers may also be added as nanotubes and nanoparticles onto the hair electrode base 210 to form the conductive material layers 212 and 214. Other non-limiting examples include but are not limited to spin-coating, dipping, laminating (roll-to-roll), brush coating, and spray coating, alone or in any combination.

The electrode with the conductive and non-conductive portions thus formed may be referred to as a 2-dimensional hair electrode 216, as described above. The 2-dimensional hair electrode 216 may also be referred to as a 2-dimensional bristle electrode. The 2-dimensional hair electrode 216 (or 2-dimensional bristle electrode 216) may have any shape, including but not limited to spherical, conical, cubical, or rectangular. The tip of the 2-dimensional hair electrode 216 may be of any shape such as spherical, conical, cubical, triangle, jagged surface, or rectangular.

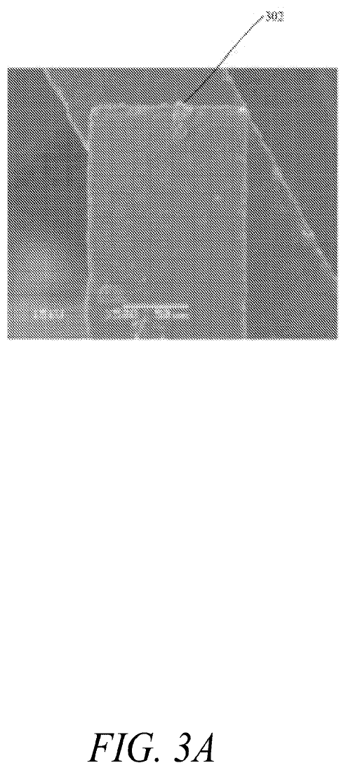

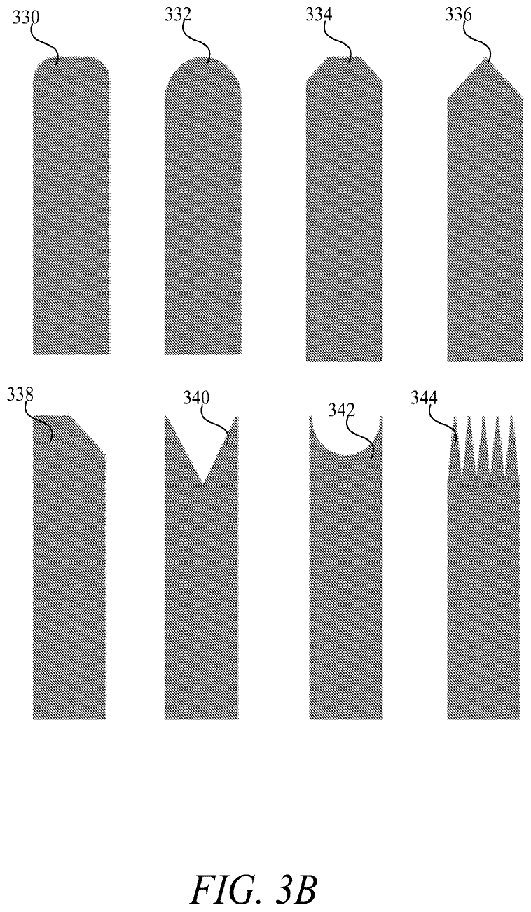

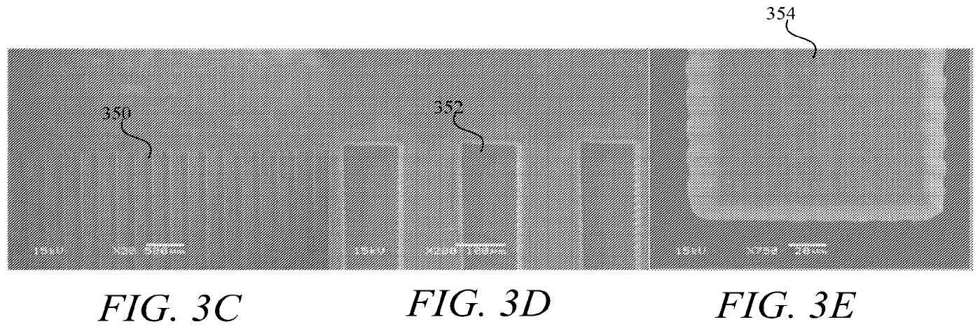

FIG. 3A-3E show example shapes of 2-dimensional hair electrode (also referred to as bristles), according to example embodiments of the present disclosure. For example, FIG. 3A shows a scanning electronic microscope (SEM) image of a 2-dimensional illustrative hair electrode 302 having a rectangular tip. The thickness of the 2-dimensional hair electrode may be 5 μm-200 μm.

FIG. 3B shows different example bristle tip end shapes, which may be used in any combination or permutation in the systems and methods described herein. In the example of FIG. 3B, the tip ends are shown at magnification as one 3-dimensional hair electrode, with the end or tip shaped as shown. As shown, FIG. 3B illustrates bristle tip end shapes 330-344. For example, bristle tip end shape 330 may correspond to a bristle tip end having a flat top but rounded edges. Bristle tip end shape 332 may correspond to a bristle tip end having a rounded tip. Bristle tip end shape 334 may correspond to a bristle tip end having two corners cut off in a three sided angular shape. Bristle tip end shape 336 may correspond to a pointed tip shape. Bristle tip end shape 338 may correspond to an angular shape with one corner cut off. Bristle tip end shape 340 may correspond to a triangular shape cut out of the end with two points left at the tip. Bristle tip end shape 342 may correspond to a rounded or circular shape cut from the tip leaving two points at the tip. Bristle tip end shape 344 may correspond to five saw-tooth shaped edges cut into the tip. In other examples not shown, two, three, four, six, or any other number of saw-toothed shaped edges may be formed at the tip as shown with bristle tip end shape 344 as a non-limiting example. In some examples, these or other shapes may be combined in the tip shape, the example shapes of FIG. 3B is non-limiting.

FIG. 3C shows an example bristle tips 350 under microscopic enlargement, according to example embodiments. As shown in FIG. 3C, bristle tips 350 are imaged at 30× imaging. FIG. 3D shows example bristle tips 352 under microscopic enlargement, according to example embodiments. As shown in FIG. 3D, multiple bristle tips 352 can be seen at 200× times imaging. FIG. 3E shows example bristle tips 354 under microscopic enlargement, according to example embodiments. As shown in FIG. 3E, a bristle tip 354 can be seen at 750×.

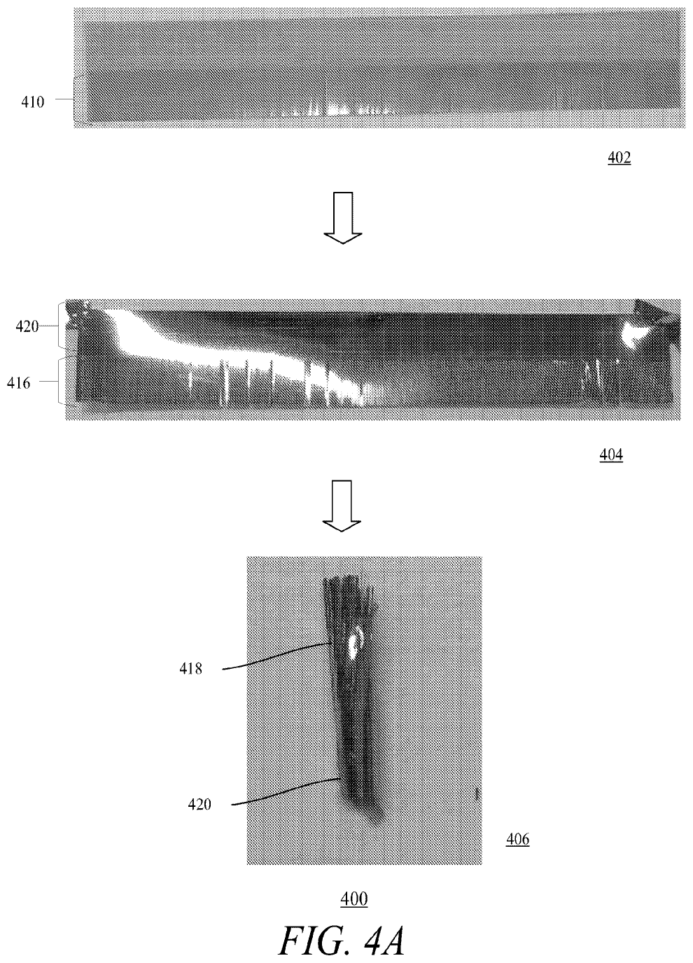

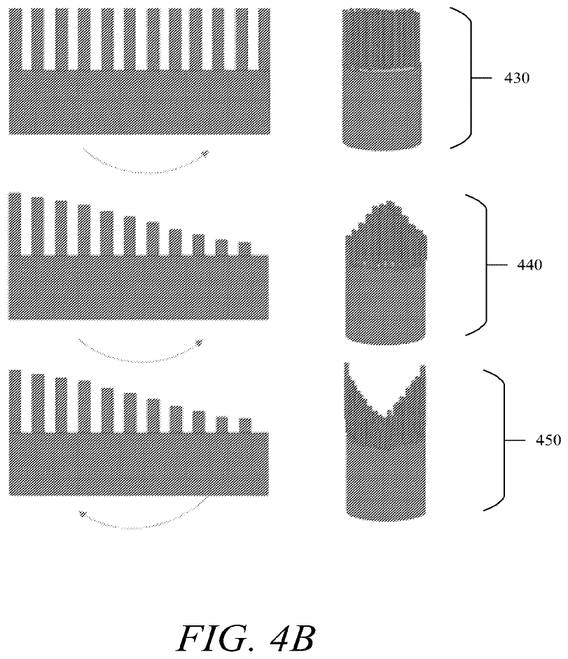

In some embodiments, a plurality of 2-dimensional hair electrodes 216 may be grouped together to form a 3-dimensional electrode (several examples of 3-dimensional electrodes are described below). In some embodiments, the 2-dimensional hair electrodes 216 may be formed in batches, where a batch may be used to form a 3-dimensional electrode (e.g., by bunching a batch of the 2-dimensional electrodes), as described with reference to FIGS. 4A-4B below.

FIG. 4A shows an illustrative process 400 of generating a 3-dimensional electrode, according to some embodiments of the present disclosure. The process 400 is illustrated may include steps 402, 404, and 406, which are provided as just examples and should not be considered limiting. Processes with alternative, additional, or fewer number of steps are to be considered within the scope of this disclosure. Furthermore, the steps 402, 404, and 406 are merely for identification and is meant neither to convey a limitation to the shown discrete steps nor a limitation to a sequence of shown steps.

At step 402, a batch of hair electrode bases 410 is formed. For instance, the hair electrode bases 410 may be formed of a synthetic material such as a polymer. As shown, a sheet of the synthetic material may be subjected to a shaping mechanism (e.g., a laser cutting) to cut the lower portion of the synthetic material into several strands. Each strand may form a 2-dimensional hair electrode base 410 (such a 2-dimensional hair electrode base 210 shown in FIG. 2A), the 2-dimensional hair electrode base being non-conductive and configured to provide structural integrity and desired mechanical properties to the corresponding 2-dimensional electrode.

At step 404, the batch of hair electrode bases 410 may receive a coating of a conductive material. For example, a portion 420 of the synthetic sheet 422 without the hair electrode bases 410 may be taped (e.g., using a masking tape), and the untaped portion may receive the conductive coating, e.g., through metal sputtering. The sheet 422 may then be turned over and the portion on the other side of portion 420 may be taped, and the untaped portion may receive the conductive coating. Such double-sided coating may generate the batch of 2-dimensional hair electrodes 416, each with non-conductive inner material and conductive outside layer.

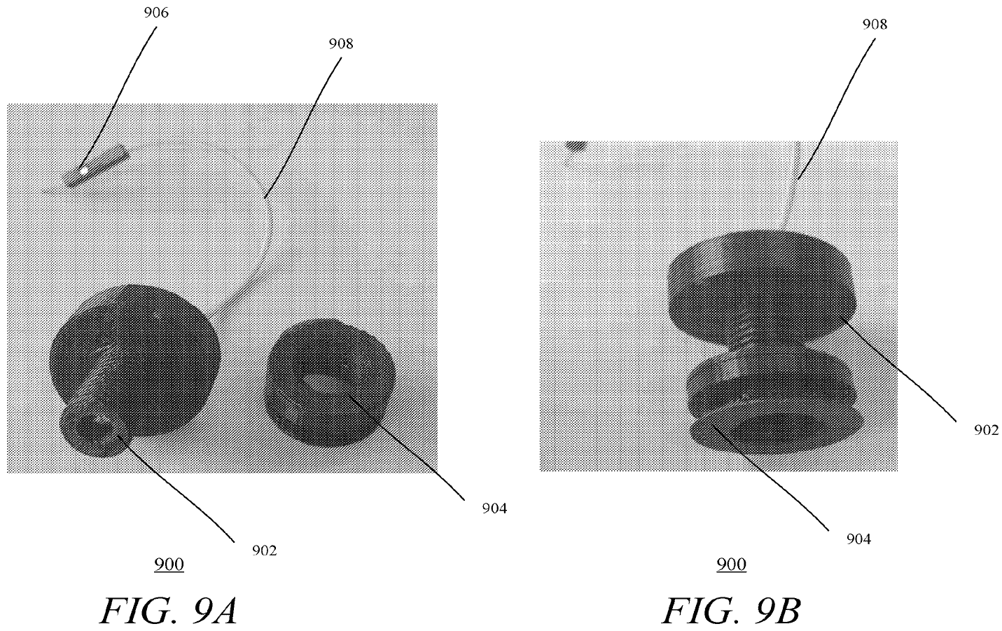

At step 406, the batch of 2-dimensional hair electrodes 416 may be rolled to form a 3-dimensional electrode 418. The 2-dimensional hair electrodes 416 within the 3-dimensional electrode 418 may be held together by a holder (described in detail with reference to FIGS. 9-12 below). The holder may be made of any type of material such as a thermal plastic elastomer, elastomer, polymer, 2-dimensional printed structure, 3-dimensional printed structure, a cap structure, or a fabric structure. In some embodiments, the rolling step 406 may take place prior to the coating step 404.

It should be understood that the aforementioned process 400 of generating the 3-dimensional electrode 418 is just an example, and other types of processes should also be considered within the scope of this disclosure. Another type of 3-dimensional electrode may be formed by rolling conductive films-the conductive films forming the individual flexible electrodes (e.g., as an alternative to the 2-dimensional hair electrodes). A 3-dimensional electrode may also be formed by a rolled electrode with a flexible substrate (e.g., not necessarily shaped into hair like structure). The rolled flexible substrate may then be coated with a conductive material. Alternatively, the 3-dimensional electrode may be formed by rolled flexible substrate electrodes already having a metal coating.

In some other non-limiting examples, the “2-dimensional” flat electrode bristles may be shaped in other ways including but not limited to circular, spherical, or other 3-dimensional structures or patterns.

As an alternative to rolling or any other type of batching, a 3-dimensional electrode may also be generated through injection molding. For instance, the 3-dimensional structure of the 3-dimensional electrode may directly be generated through one or more steps of injection molding. Alternatively, the 3-dimensional electrode may be generated through 3-dimensional printing, which may print different layers eventually forming the 3-dimensional electrode.

Using one or more of the above techniques, different types of 3-dimensional electrodes may be generated. Some examples of the 3-dimensional electrodes are described below.

FIG. 4B shows examples of differently shaped or sized bristles (i.e., hair electrodes) rolled into different shapes, according to example embodiments of the present disclosure. Such examples may include, but are not limited, to flat bristles rolled into a flat shape 430, descending height bristles rolled into a pointed shape 440, and descending bristles rolled into an inverted wedge shape 450. Different shapes could be made by rolling varyingly sized bristles, the examples of FIG. 4B are not intended to be limiting.

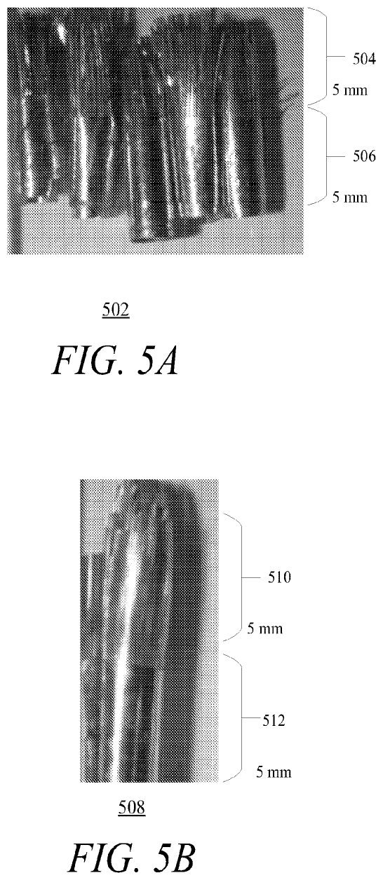

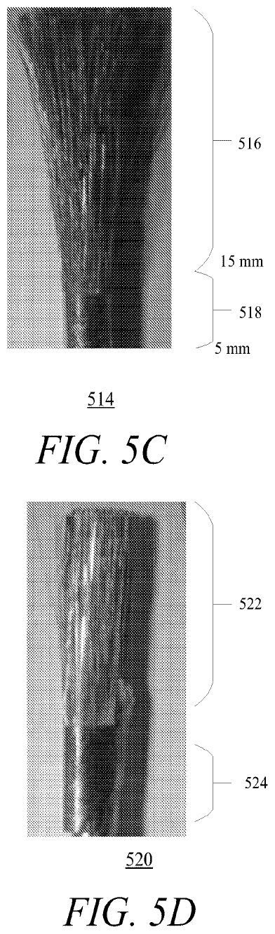

FIGS. 5A-5D show illustrations of 3-dimensional electrodes, according to example embodiments of the present disclosure. FIG. 5A shows a 3-dimensional electrode 502, according to example embodiments. As shown, 3-dimensional electrode 502 may include a batch of 2-dimensional hair electrodes 504 and a holder 506 holding the batch of 2-dimensional hair electrodes 504. As shown, the length of the 2-dimensional hair electrodes may be 5 mm and the height of the holder 506 may be about 5 mm, thereby making the total length of the 3-dimensional electrode to be about 10 mm. In the shown example, the width of each 2-dimensional hair electrode may be about 140 μm. In some examples, a gap distance between the individual 2-dimensional hair electrodes 504 may be about 90 μm, about 50 μm, about 10 μm or other example gaps.

FIG. 5B shows a 3-dimensional hair electrode 508, according to example embodiments. As shown, 3-dimensional hair electrode 508 may include a batch of 2-dimensional hair electrodes 510 and a holder 512 holding the batch of 2-dimensional hair electrodes 510. As shown, the length of the batch of the 2-dimensional hair electrodes 510 may be 5 mm and the height of the holder 512 holding the batch of 2-dimensional hair electrodes 510 may be 5 mm, thereby making the total length of the 3-dimensional electrode 508 to be 10 mm. The width of each 2-dimensional hair electrode 510 may be 140 μm. Other example widths of each 2-dimensional hair electrode 510 may be 25 μm, 50 μm, 75 μm and/or 125 μm in any combination or permutation as well as similar or the same dimension of all hair electrodes in one system example.

FIG. 5C shows a 3-dimensional electrode 514, according to example embodiments. As shown, 3-dimensional electrode 514 may include a batch of 2-dimensional hair electrodes 516 and a holder 518 holding the batch of 2-dimensional hair electrodes 516. As shown, the length of the batch of the 2-dimensional hair electrodes 510 may be 15 mm and the height of the holder 518 holding the batch of 2-dimensional hair electrodes 510 may be 5 mm thereby making the length of the 3-dimensional electrode to be 20 mm. The width of each of each 2-dimensional hair electrode 516 may be 140 μm.

FIG. 5D shows a 3-dimensional electrode 520, according to example embodiments. As shown, 3-dimensional electrode 520 may include a batch of 2-d hair electrodes 522 and a holding 524 holding the batch of 2-dimensional hair electrodes 522. Additional design options may include electrodes having hair bristles of different lengths with a staggered design. In some examples, different types may have different numbers of hair electrodes in one example.

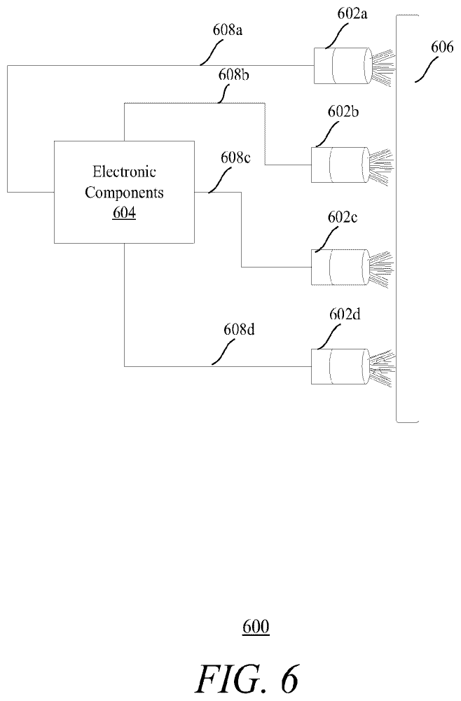

FIG. 6 shows an illustrative circuit 600 using the 3-dimensional electrodes, according to example embodiments of the present disclosure. It should be understood that the circuit 600 is merely for illustration and should not be considered limiting. Circuits with additional, alternative, and fewer number of components should be considered within the scope of this disclosure. The circuit 600 may include 3-dimensional electrodes 602a-602d (commonly referred to as a 3-dimensional electrode 602 and collectively referred to as 3-dimensional electrodes 602), electronic components 604, communication links 608a-608d (commonly referred to a communication link 608 and collectively referred to as communication links 608) between the 3-dimensional electrodes and the electronic components 604, and live tissue 608 (e.g., human skin) that the 3-dimensional electrodes are attached to.

Each of the 3-dimensional electrodes 602 may detect and measure electrical signals in the live tissue 606. The measured electrical signals may be transmitted to the electronic components through the corresponding communication links 608. The communication links 608 may include wired and/or wireless communication links. In some embodiments, the measured electrical signal may be amplified. The amplification may be within the electrodes 602, at one or more points in the communication links 608, and/or the electronic components 604. In some embodiments, the electrodes 602 may be passive electrodes, not necessarily providing amplification or other electronic functionality.

The electronic components 604 may further process and analyze the electrical signals detected by the 3-dimensional electrodes 602 and amplified at one or more points. For example, the electronic components 604 may include a digital processor that may convert the signal into a graphical output (e.g., a heartbeat pattern of an ECG). The electronic components 604 may further comprise a storage (e.g., hard disk, solid state drive) to store the measured electrical signals and/or patterns extracted therefrom. The electronic components 604 may further have communication components to communicate the measured electrical signals and/or patterns extracted therefrom to other external components (e.g., communicating to a remote computer through a network).

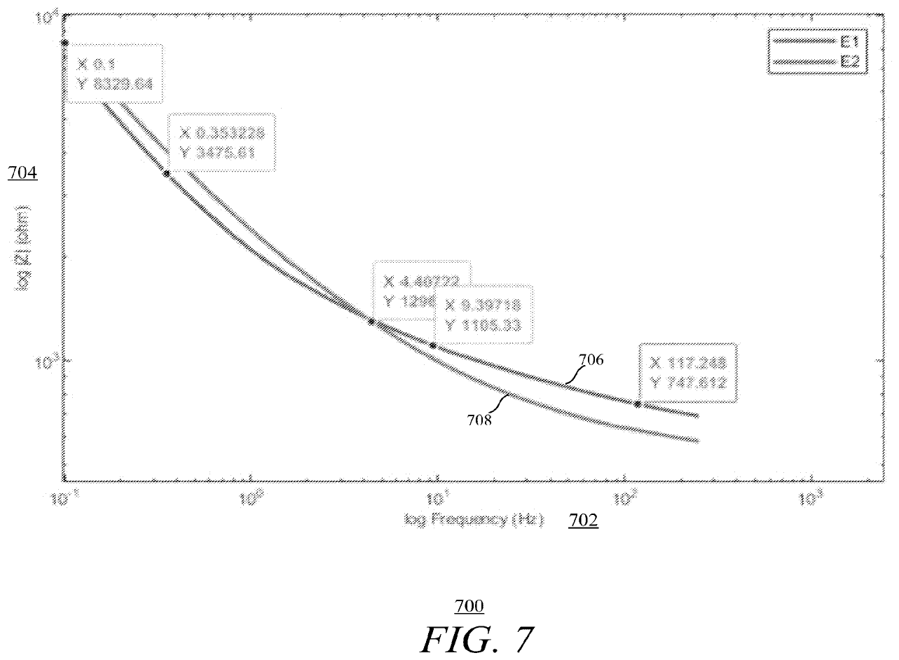

FIG. 7 shows a graph 700 impedance characteristics of an illustrative 3-dimensional electrode compared to impedance characteristics of a conventional wet electrode, according to some embodiments of the present disclosure. As shown, the horizontal axis 702 has a log of the frequency sweep of the measurement and the vertical axis 704 has a log of the impedance measured on ohms. Line graph 706 shows the impedance characteristics of a 3-dimensional electrode and line graph 708 shows the impedance characteristics of a conventional wet electrode. As it can be seen, the performance of the 3-dimensional electrode is compared to the performance of the wet electrode, thereby indicating that the 3-dimensional electrode, formed according to some embodiments of this disclosure, achieves the desired clinical performance level.

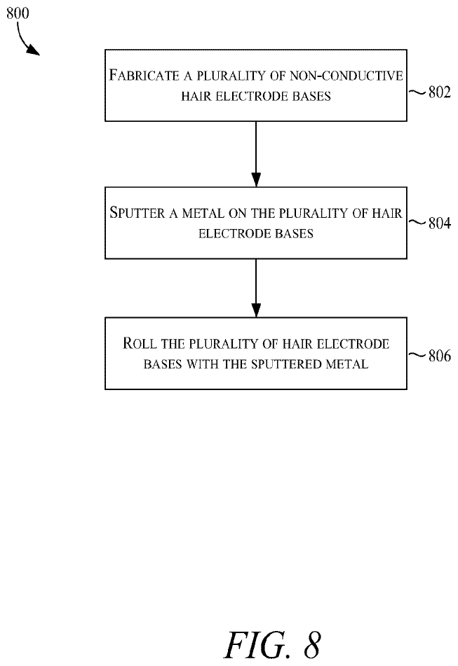

FIG. 8 shows a flow diagram of an illustrative method 800 of manufacturing a 3-dimensional electrode, according to example embodiments of the present disclosure. The method 800 is illustrated includes steps 802, 804, and 806, which are provided as just examples and should not be considered limiting. Methods with alternative, additional, or fewer number of steps are to be considered within the scope of this disclosure. Furthermore, the steps 802, 804, and 806 are merely for identification and is meant neither to convey a limitation to the shown discrete steps nor a limitation to the shown sequence of the steps.

At step 802, a plurality of non-conductive hair electrode bases (to form the 2-dimensional hair electrodes) may be fabricated. The fabrication may be through any kind of mechanism. For instance, a non-conductive substrate (e.g., a polymer) may be laser sculpted to form the non-conductive hair bases.

At step 804, metal (e.g., gold) may be sputtered to the plurality of hair electrode bases. The metal may form a conductive portion of the 2-dimensional hair electrodes. It should be understood that sputtering the metal is just an example of adding a conductive portion of the 2-dimensional hair electrodes. Alternatives of adding the conductive portion include dipping the non-conductive hair electrode bases into a metal paste, thermally depositing the metal, electronically depositing the metal, etc. Furthermore, conductive polymers may be used instead of the metal.

At step 806, the plurality of hair electrode bases with the sputtered metal may be rolled to form the 3-dimensional electrode. The rolling is also just an example, and the 3-dimensional electrode may be formed through other techniques such as 3-dimensional printing, injection molding, etc.

FIGS. 9A-9B show an example holder 900 for a 3-dimensional electrode, according to example embodiments of the present disclosure. It should be understood that the holder 900 is intended as an example and should not be considered limiting. Holders with additional, alternate, or fewer number of components should be considered within the scope of this disclosure.

The holder 900 is designed fit into existing EEG cap sockets, therefore alterations to the existing EEG cap sockets may not be required to accommodate the embodiments disclosed herein. As shown, the holder 900 may include a screw holder 902 and a fastening member 904. The screw holder 902 may have a hollow middle portion that may be used to pass a wire 908, which in turn may be attached to a 3-dimensional electrode 906. FIG. 9A shows the screw holder 902 being disconnected from the fastening member 904 and FIG. 9B shows the screw holder 902 being connected to the fastening member 904.

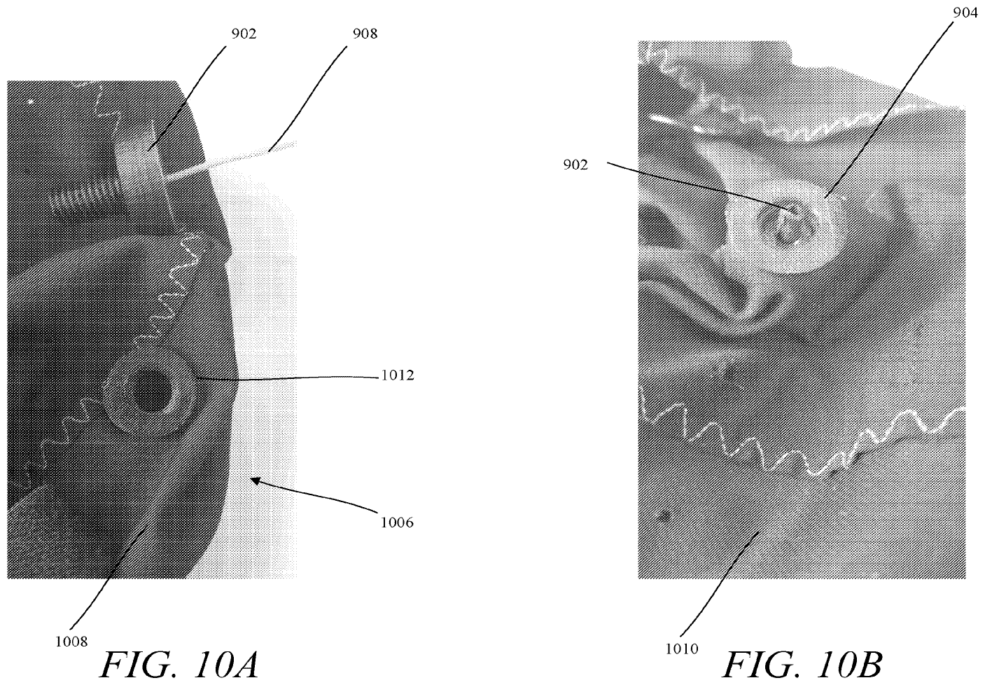

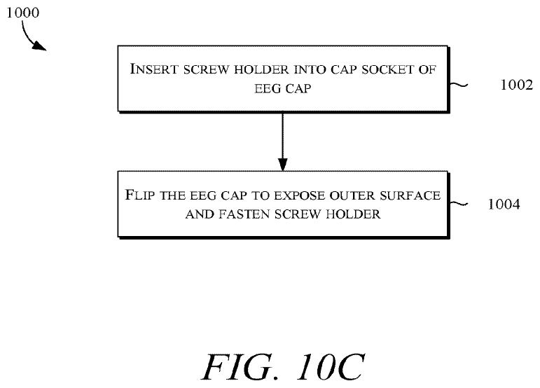

FIGS. 10A-10B show example views of using a holder with the 3-dimensional electrode, according to example embodiments of the present disclosure. Particularly, FIG. 10A shows the screw holder 902 and an inside surface 1008 of an EEG cap 1006 and FIG. 10B shows the fastening member 904 on an outside surface 1010 of the EEG cap 1006.

FIG. 10C shows an example method 1000 of using the holder with the 3-dimensional electrode, according to example embodiments of this disclosure. The process 1000 is shown to be comprising steps 1002 and 1004; it should however be understood that these steps are just shown as examples and ease of explanation and therefore should not be considered limiting.

At step 1002, the screw holder 902 may be inserted into a cap socket 1012 of an EEG cap 1006. The screw holder 902 may be inserted from the surface that interfaces the human head, i.e., the shown surface at step 1002 is the inside surface 1008 of the EEG cap 1006. The screw holder 902 may already have the wire 908 passing through its hollow interior, where the wire has a 3-dimensional electrode attached at the end (e.g., as shown in FIG. 9 above).

At step 1004, the EEG cap 1006 may be flipped over to expose an outer surface 1010 (i.e., the surface not touching the human head). The fastening member 904 may be fastened to the screw holder 902, thereby tightening the holder 900 on the EEG cap 1006.

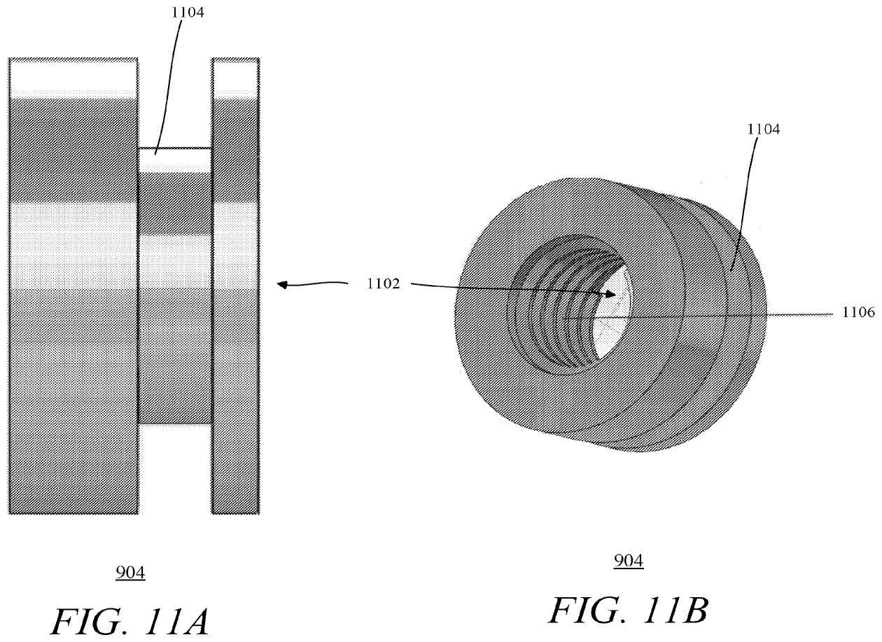

FIGS. 11A-11B show an example fastening member 904 of a holder of a 3-dimensional electrode, according to example embodiments of the present disclosure. Particularly, FIG. 11A shows a side view and FIG. 11B shows a perspective view (right image). As shown, the fastening member 904 may include a mating portion 1104 that mates with the socket 1012 of the EEG cap 1006. For example, the socket 1012 may be made of a stretchable fiber and/or rubber-like material that may be stretched to be accommodated into the mating portion 1104. The fastening member 904 may further include threads configured to receive threads of the screw holder 902. That is, the screw holder may be accommodated into the hollow center 1102 of the fastening member 904.

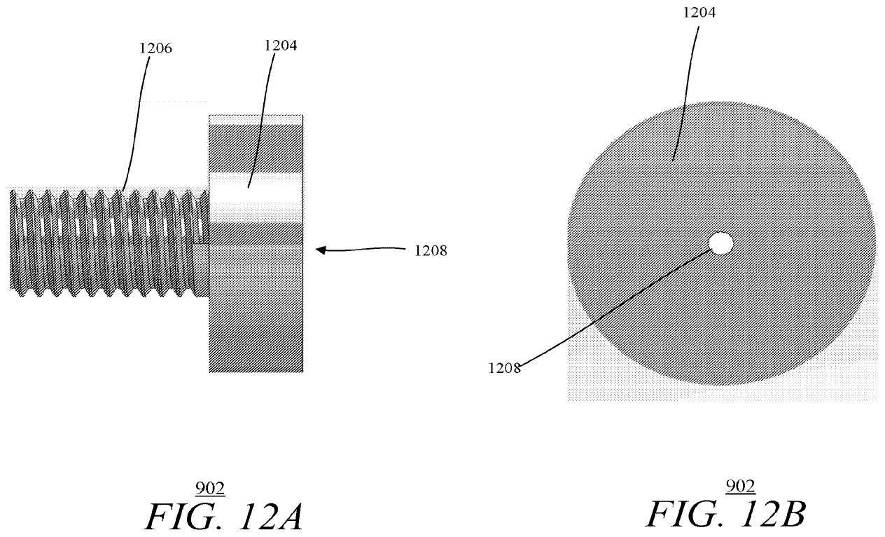

FIGS. 12A-12B show an example screw holder 902 of a holder of a 3-dimensional electrode, according to example embodiments of the present disclosure. Particularly, FIG. 12A shows a side view and FIG. 12B shows a top view (right image). As shown, the screw holder 902 may include a screw head 1204 and a threaded stem 1206. The threaded steam 1206 is configured to be threaded to threads 1106 of the fastening member 904 (shown in FIGS. 11A-11B). As shown in the top view, a hollow portion 1208 allows a wire with 3-dimensional electrode attached thereon to be passed through.

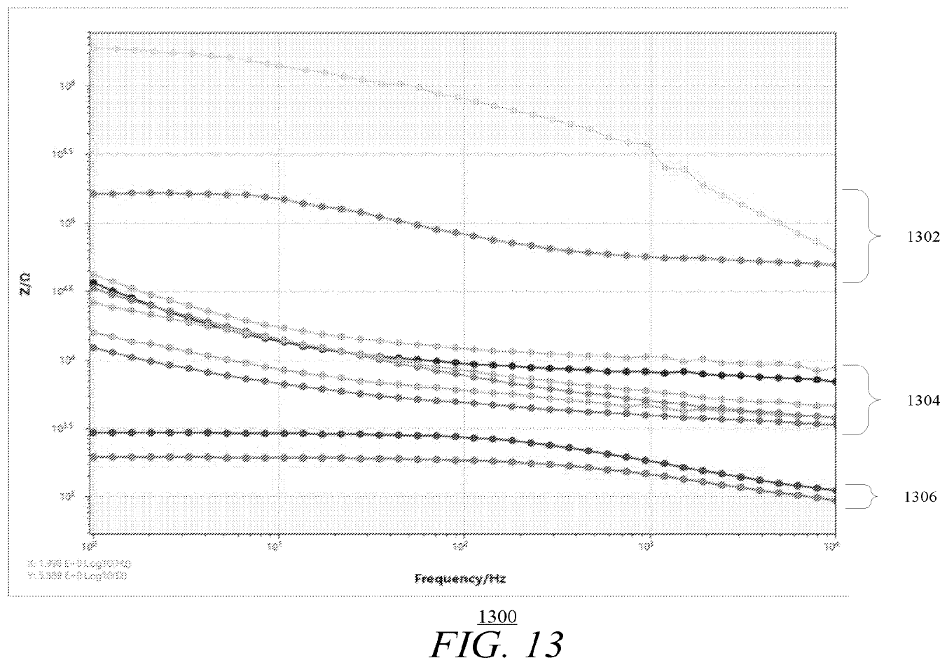

FIG. 13 shows an example chart 1300 illustrating a comparison of the 3-dimensional electrodes, according to example embodiments of the present disclosure, with conventional dry and wet electrodes. Within the example chart 1300, lines 1302 show the frequency-impendence of dry electrodes, lines 1304 show frequency-impedance of the 3-dimensional electrodes, and lines 1306 show frequency-impendence of conventional AgCl wet electrodes. As shown, the 3-dimensional electrodes are significantly better than the dry electrodes and comparable to the industry standard AgCl wet electrodes.

Additional examples of the presently described method and device embodiments are suggested according to the structures and techniques described herein. Other non-limiting examples may be configured to operate separately or can be combined in any permutation or combination with any one or more of the other examples provided above or throughout the present disclosure.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A flexible electrode for a clinical equipment and configured to detect electrical signals from a live tissue, the flexible electrode comprising:
a plurality of hair electrodes, wherein each of the hair electrodes includes,
a non-conductive and flexible hair electrode base defining a structure of the hair electrode; and
a gold outer layer covering the electrode base, the gold outer layer forming a conductive portion of the hair electrode and configured to detect the electrical signals from the live tissue.

2. The flexible electrode of claim 1, wherein the hair electrode has a shape comprising at least one of spherical, conical, cubical, or rectangular.

3. The flexible electrode of claim 1, wherein a tip of the hair electrode has a shape comprising at least one of spherical, conical, cubical, or rectangular.

4. The flexible electrode of claim 1, hair electrode base comprises a flexible pillar.

5. The flexible electrode of claim 1, wherein the hair electrode base comprises a flexible tube.

6. The flexible electrode of claim 1, wherein the hair electrode base is formed by at least one of a polymer, cellulose, chitin, chitosan, synthetic flexible fiber, microfiber, nanotube, nanofiber, or parylene.

7. The flexible electrode of claim 1, wherein the gold outer layer is formed by at least one of:
sputtering gold on the hair electrode base,
thermally depositing gold on the hair electrode base,
electrochemically depositing gold on the hair electrode base,
dipping the hair electrode base on a gold metal paste,
adding gold nanotubes on the hair electrode base, or
adding gold nanoparticles on the hair electrode base.

8. The flexible electrode of claim 1, wherein the flexible electrode is formed by rolling together the plurality of hair electrodes.

9. The flexible electrode of claim 8, wherein the rolling comprises at least one of:
rolling a conductive film where the plurality of hair electrodes are attached to,
rolling a flexible substrate attached to the plurality of hair electrodes prior to an application of the gold outer layer, or
rolling a flexible substrate attached to the plurality of hair electrodes after the application of the gold outer layer.

10. The flexible electrode of claim 1, wherein the flexible electrode is formed by an injection molding.

11. The flexible electrode of claim 1, wherein the flexible electrode is formed by a 3-dimensional printing.

12. The flexible electrode of claim 1, wherein each of the hair electrodes is formed by at least one of: laser cutting, laser marking, water jet cutting, diamond cutting, or injection molding.

13. The flexible electrode of claim 1, further comprising a holder holding the plurality of hair electrodes together, the holder formed by at least one of: thermal plastic elastomer, elastomer, polymer, 2-dimensional printed structure, 3-dimensional printed structure, or fabric.

14. The flexible electrode of claim 1, further comprising an amplifier configured to amplify the detected electrical signals.

15. The flexible electrode of claim 1, wherein the flexible electrode forms a passive electrode.

16. The flexible electrode to claim 1, configured to transmit the detected electrical signals wirelessly.

17. The flexible electrode of claim 1, configured to transmit the detected electrical signals through a wire.

18. A flexible electrode configured for a clinical equipment and configured to detect electrical signals from a live tissue, the flexible electrode comprising:
a plurality of hair electrodes, wherein each of the hair electrodes comprises:
a non-conductive and flexible hair electrode base defining a structure of the hair electrode; and
a conductive polymer outer layer covering the electrode base, the conductive polymer outer layer forming a conductive portion of the hair electrode and configured to detect the electrical signals from the live tissue.

19. The flexible electrode of claim 18, wherein the conductive polymer outer layer is formed by at least one of:
electrochemically depositing a conductive polymer on the hair electrode base,
dipping the hair electrode on a conductive polymer paste,
adding conductive polymer nanotubes on the hair electrode base, or
adding conductive polymer nanoparticles on the hair electrode base.

20. A method of manufacturing a flexible electrode for a clinical equipment and configured to detect electrical signals from a live tissue, the flexible electrode comprising a plurality of hair electrodes, the method comprising:
fabricating a plurality of non-conductive hair electrode bases for the plurality of hair electrodes;
sputtering a metal on the plurality of hair electrode bases to form conductive portions of the plurality of hair electrodes; and
rolling the plurality of hair electrode bases with the sputtered metal to form the flexible electrode.
